(19) United States
(12) Patent Application Publication
(10) Pub. No.: US 2019/0384013 A1
(43) Pub. Date: Dec. 19, 2019

(54) OPTICAL MODULE, ENDOSCOPE AND MANUFACTURING METHOD OF OPTICAL MODULE

(71) Applicant: OLYMPUS CORPORATION, Tokyo (JP)

(72) Inventor: Yusuke NAKAGAWA, Ina-shi (JP)

(73) Assignee: OLYMPUS CORPORATION, Tokyo (JP)

(21) Appl. No.: 16/533,888

(22) Filed: Aug. 7, 2019

Related U.S. Application Data
(63) Continuation of application No. PCT/JP2017/005698, filed on Feb. 16, 2017.

(51) Int. Cl. G02B 6/38; A61B 1/04; G02B 6/40

(52) U.S. Cl. CPC G02B 6/3874; A61B 1/04; G02B 6/3839; G02B 6/3861; G02B 6/406

(57) ABSTRACT

An optical module includes: an optical fiber; a ferrule with an insertion hole where the optical fiber is inserted; a sleeve with a through hole where the ferrule is inserted; a light emitting element; and a wiring board having a first principle surface where the optical element is disposed and a second principle surface where the sleeve is disposed. The sleeve has a deformation range where an outer surface is concave and an inner surface defining the through hole is convex, the inner surface of the deformation range being in contact with the ferrule. The sleeve is a metal cylinder with a resin provided to fill a space between the metal cylinder and the ferrule.

### CROSS REFERENCE TO RELATED APPLICATION

This application is a continuation application of PCT/JP2017/005698 filed on Feb. 16, 2017, the entire contents of which are incorporated herein by this reference.

### BACKGROUND OF INVENTION

### 1. Field of the Invention

The present invention relates to an optical module, an endoscope including the optical module, and a production method of the optical module, the optical module including an optical element that emits or receives an optical signal, an optical fiber that transmits the optical signal, a ferrule with an insertion hole where the optical fiber is inserted, and a wiring board having a first principle surface where the ferrule is disposed and a second principle surface where the optical element is mounted.

### 2. Description of the Related Art

An endoscope includes an elongated flexible insertion portion at a distal end portion that is provided with an image pickup device such as a CCD. Use of an image pickup device with a large number of pixels in an endoscope has recently been studied. In a case of using an image pickup device with a large number of pixels, an amount of signal from the image pickup device to a signal processor increases. Accordingly, instead of an electric signal transmission such as transmission of an electric signal through a metal wiring, an optical signal transmission such as transmission of an optical signal through a thinner optical fiber is preferable. For optical signal transmission, an E/O optical module, i.e., electricity-to-light converter, that converts an electric signal into an optical signal and an O/E optical module, i.e., light-to-electricity converter, that converts an optical signal into an electric signal are usable.

For example, an optical module includes an optical element, an optical fiber, a ferrule with the optical fiber inserted, and a wiring board having a first principle surface where the ferrule is disposed and a second principle surface where the optical element is mounted. To produce the optical module, the ferrule with the optical fiber inserted is bonded to the wiring board where the optical element is disposed, which is not easy. In other words, the ferrule and the wiring board need to be held with the optical fiber and the optical element being positioned each other until an adhesive cures.

Japanese Patent Application Laid-Open Publication No. 5-164941 discloses an optical connector including an optical fiber that is inserted in a sleeve made of an elastic material so that the optical fiber is removably attached.

Japanese Patent Application Laid-Open Publication No. 2015-49374 discloses a ferrule-attached optical fiber, which is an optical fiber inserted in a ferrule including a metal cylinder and fixed by crimping the metal cylinder.

### SUMMARY OF THE INVENTION

According to an embodiment, an optical module includes: an optical fiber configured to transmit an optical signal; a ferrule with an insertion hole in which the optical fiber is inserted; a sleeve with a through hole in which the ferrule is inserted; an optical element configured to emit or receive the optical signal; and a wiring board having a first principle surface and a second principle surface, the optical element being disposed on the first principle surface, the sleeve being disposed on the second principle surface. The sleeve has a deformation range where an outer surface is concave and an inner surface defining the through hole is convex, the inner surface of the deformation range being in contact with the ferrule, and the sleeve is a metal cylinder with a resin provided to fill a space between the metal cylinder and the ferrule.

According to another embodiment, an endoscope includes: an insertion portion; and an optical module provided at the insertion portion, the optical module including: an optical fiber configured to transmit an optical signal; a ferrule with an insertion hole in which the optical fiber is inserted; a sleeve with a through hole in which the ferrule is inserted; an optical element configured to emit or receive the optical signal; and a wiring board having a first principle surface and a second principle surface, the optical element being disposed on the first principle surface, the sleeve being disposed on the second principle surface. The sleeve has a deformation range where an outer surface is concave and an inner surface defining the through hole is convex, the inner surface of the deformation range being in contact with the ferrule, and the sleeve is a metal cylinder with a resin provided to fill a space between the metal cylinder and the ferrule.

According to still another embodiment, a production method of an optical module, the optical module including: an optical fiber configured to transmit an optical signal; a ferrule with an insertion hole; a sleeve being a metal cylinder with a through hole; an optical element configured to emit or receive the optical signal; and a wiring board having a first principle surface and a second principle surface, the optical element being disposed on the first principle surface, the sleeve being disposed on the second principle surface, the method includes: disposing the optical element and the sleeve on the wiring board; fixing the optical fiber to the ferrule after the optical fiber is inserted in the through hole of the ferrule; inserting the ferrule in which the optical fiber is inserted and an uncured adhesive in the through hole of the sleeve; forming a deformation range where an outer surface is concave and an inner surface defining the through hole is convex by plastic deformation of the sleeve; fixing the ferrule to the sleeve by bringing the ferrule into contact with the inner surface of the deformation range; and curing the adhesive.

### BRIEF DESCRIPTION OF THE DRAWINGS

FIG. 1 is an exploded view of an optical module according to a first embodiment;

FIG. 2 is a sectional view of the optical module according to the first embodiment;

FIG. 3 is a top view of the optical module according to the first embodiment;

FIG. 4 is a flowchart for explaining a production method of the optical module according to the first embodiment;

FIG. 5 is a sectional view of an optical module according to a modification 1 of the first embodiment;

FIG. 6 is a sectional view of an optical module according to a modification 2 of the first embodiment;

FIG. 7 is a sectional view of an optical module according to a modification 3 of the first embodiment;

FIG. 8 is a perspective view of an optical module according to a modification 4 of the first embodiment;

FIG. 9 is a perspective view of an optical module according to a modification 5 of the first embodiment;

FIG. 10 is a perspective view of an optical module according to a modification 6 of the first embodiment;

FIG. 11 is a top view of an optical module according to a second embodiment;

FIG. 12 is a sectional view of an optical module according to the second embodiment taken along a line XII-XII in FIG. 11;

FIG. 13 is a top view of an optical module according to a modification 1 of the second embodiment;

FIG. 14 is a sectional view of an optical module according to a modification 2 of the second embodiment; and

FIG. 15 is a perspective view of an endoscope according to a third embodiment.

### DETAILED DESCRIPTION OF THE PREFERRED EMBODIMENT(S)

### First Embodiment

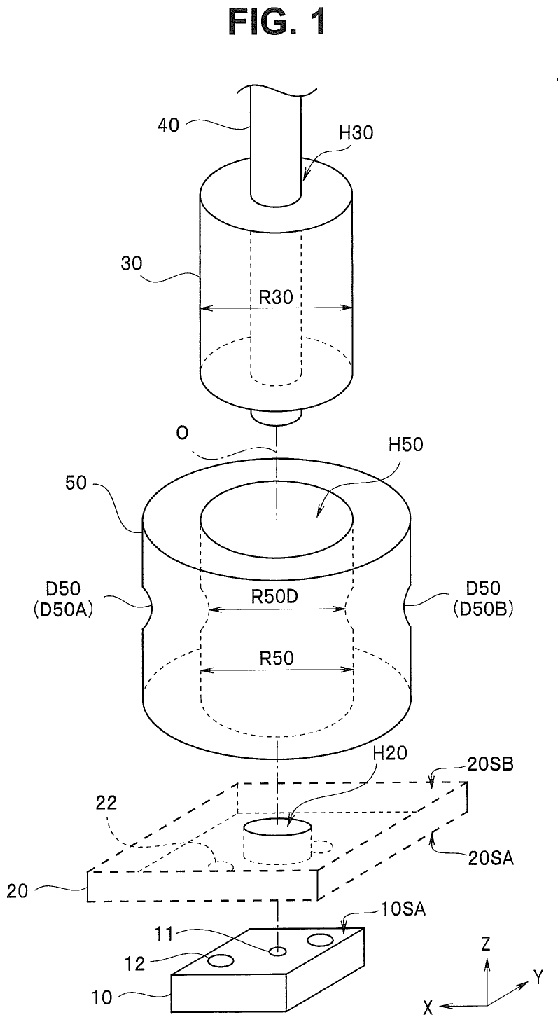

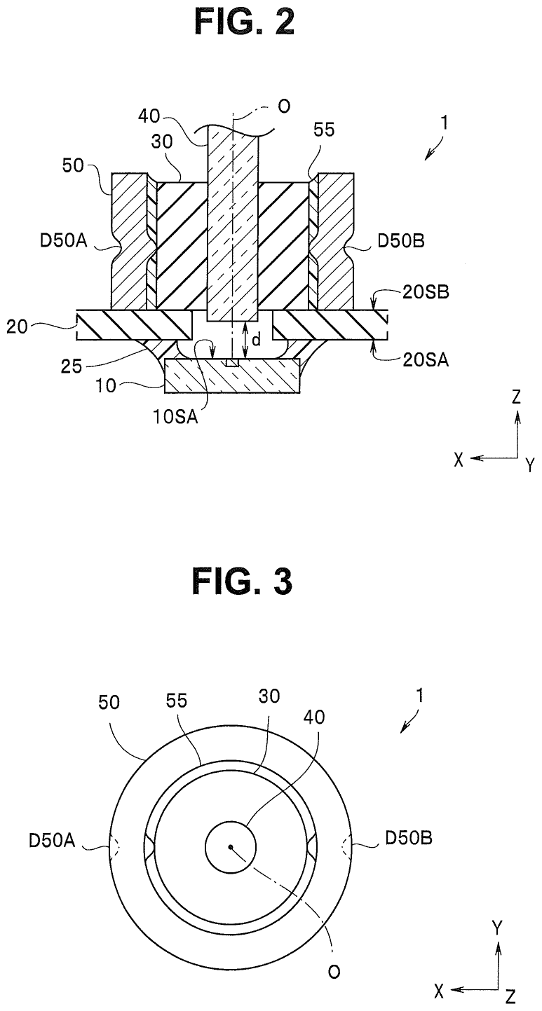

As shown in FIGS. 1 and 2, an optical module 1 according to the present embodiment includes a light emitting element 10, a wiring board 20, a ferrule 30 with an insertion hole H30, an optical fiber 40, and a sleeve 50 with a through hole H50.

Regarding the following description, note that drawings based on embodiments are so schematic that a relationship between a thickness and a width of each part, a thickness ratio and a relative angle between parts, etc. are different from actual ones, and mutual dimensional relationships and ratios among some parts may be different from one drawing to another drawing. In addition, some components may not be shown. Furthermore, in describing each of a plurality of components with the same function, the last one character of a reference sign may be omitted.

The light emitting element 10, which is a VCSEL (vertical cavity surface emitting laser), has a light emitting surface 10SA, i.e., a front surface, provided with a light emitting portion 11, i.e., an optical element portion, that emits an optical signal. For example, the light emitting element 10 is an ultracompact element with a dimension of 235 μm×235 μm in plan view, including the 10-μm diameter light emitting portion 11 and two 70-μm diameter external terminals 12 that supply the drive signal to the light emitting portion 11 on the light emitting surface 10SA.

The wiring board 20, which is in the form of a plate, has a first principle surface 20SA and a second principle surface 20SB. The light emitting element 10 is disposed on the first principle surface 20SA and a ferrule 30, which is inserted in the through hole H50 of the sleeve 50, is disposed on the second principle surface 20SB. In other words, two connection electrodes 22, which are bonded to the external terminals 12 of the light emitting element 10, are disposed on the first principle surface 20SA. The drive signal is supplied to each of the connection electrodes 22 through a wiring (not shown).

The wiring board 20 has a hole H20, which functions as a light path for the optical signal. The wiring board 20 may be an FPC wiring board, a ceramic wiring board, a glass epoxy wiring board, a glass wiring board, or a silicon wiring board.

Note that in a case where the wiring board 20 lets light of the optical signal through, the hole H20 is not necessary. For example, in a case where the optical signal is infrared light, a silicon board that is opaque in a visible light region may be used as a wiring board with no hole H20 formed as long as the silicon board has a high light transmittance in an infrared region.

The optical fiber 40, which transmits the optical signal emitted from the light emitting element 10, includes a 62.5-μm diameter core portion that transmits light and an 80-μm diameter clad portion that covers an outer circumferential surface of the core portion.

The ferrule 30 has the insertion hole H30, which is a through hole that penetrates from an upper surface to a lower surface of the ferrule 30. In this regard, the lower surface is a surface facing the second principle surface 20SB of the wiring board 20. A distal end portion of the optical fiber 40 is inserted in the insertion hole H30 of the ferrule 30, which is in the form of a column with a length (Z-axis dimension: a dimension between the upper surface and the lower surface) of 0.5 mm, and fixed with an adhesive (not shown).

The sleeve 50, which is a metal cylinder, has an outer diameter of 1 mm and a length of 0.5 mm and the through hole H50 has an inner diameter R50 of 452 μm. Incidentally, an outer diameter R30 of the ferrule 30 is 450 μm. In other words, a 1-μm (0.001-mm) gap is formed between a side surface of the ferrule 30 inserted in the through hole H50 of the sleeve 50 and an inner surface (wall surface) of the through hole H50 of the sleeve 50. The gap between the sleeve 50 and the ferrule 30 is filled with a thermosetting resin 55 cured by a curing process, the thermosetting resin 55 being in liquid form when uncured.

The sleeve 50 includes deformation ranges D50A, D50B defined at two opposite positions with respect to a center axis of the cylindrical sleeve 50 corresponding to an optical axis O of the light emitting element 10 and the optical fiber 40. The deformation ranges D50A, D50B each have a concave outer surface and a convex inner surface, which defines the through hole H50. An inner dimension R50D of the through hole H50 at the deformation ranges D50A, D50B is the same as the outer diameter R30 of the ferrule 30.

As described later, the deformation ranges D50A, D50B are each a plastic deformation range that is plastically deformed by holding and pressing an outer surface of the sleeve 50, in which the ferrule 30 is inserted, using a holding tool such as tweezers. The inner surface of the through hole H50 of the sleeve 50 is in contact with the side surface of the ferrule 30.

The inner surface of the through hole H50 of the sleeve 50 and the ferrule 30 of the optical module 1 are firmly bonded with the thermosetting resin 55 provided to fill a space between the inner surface and the ferrule 30. The curing process of the resin 55 is performed with the ferrule 30 being temporarily fixed by the deformation ranges D50 of the sleeve 50. The optical module 1 eliminates the necessity of a special tool or the like for temporary fixation, thus exhibiting a high productivity.

The deformation ranges D50 of the sleeve 50 also hold the ferrule 30 where the optical fiber 40 is inserted. A stress F resulting from deformation of the sleeve 50 is thus not applied to the optical fiber 40. If a large stress is applied to an optical fiber, transmission properties of the optical fiber are usually lowered due to photoelasticity. However, the stress F is not applied to the optical fiber 40 according to the present embodiment, so that transmission properties of the optical module 1 are stable.

The optical module 1 is ultracompact such that the outer diameter of the sleeve 50 is, for example, 0.45 mm. Thus, by equipping the optical module 1 in, especially, an endoscope, invasiveness can be reduced.

### Production Method of Optical Module

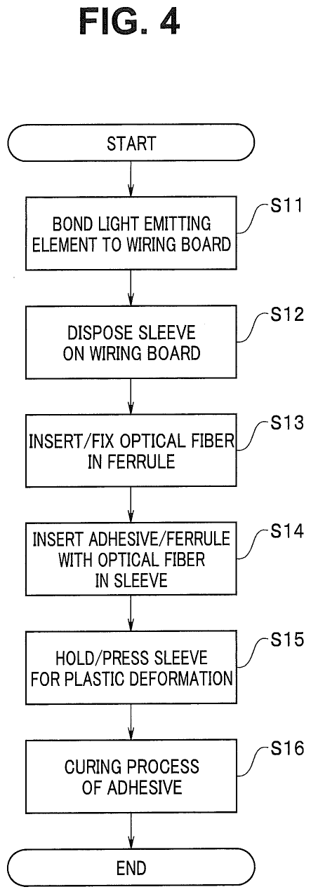

Next, a production method of the optical module 1 is explained along a flowchart in FIG. 4.

### <Step S11> Bonding of Light Emitting Element

The light emitting element 10 is flip-chip mounted on the first principle surface 20SA of the wiring board 20 with the light emitting portion 11 facing the hole H20. In other words, the external terminals 12 of the light emitting element 10 are bonded to the connection electrodes 22 of the wiring board 20.

For example, the external terminals 12 of the light emitting element 10, which are coated with a gold layer, are ultrasonically bonded to Au-stud bumps disposed on the connection electrodes 22 of the wiring board 20.

The light emitting element 10 may be mounted by printing a solder paste or the like on the wiring board 20 and disposing the light emitting element 10 at a predetermined position, and then melting the solder by a reflow process or the like. Note that the wiring board 20 may include a processing circuit for converting an electric signal transmitted from an image pickup device 90 into a drive signal for the light emitting element 10.

In addition, as shown in FIG. 2, the bonding of the wiring board 20 and the light emitting element 10 may be enhanced by a resin 25 for sidefilling, underfilling, or the like.

### <Step S12> Disposition of Sleeve

The sleeve 50 is disposed on the second principle surface 20SB of the wiring board 20 with the center axis of the sleeve 50 being in alignment with the optical axis O. The sleeve 50 may be fixed to the wiring board 20 with an adhesive or soldered to, for example, an annular conductive film of the wiring board 20.

The sleeve 50 is made of copper in the form of a plate with a thickness of 0.25 mm. To allow the sleeve 50 to stably hold the inserted ferrule 30 while being relatively easily plastically deformed, it is preferable that the sleeve 50 be made of, for example, a metal with a Vickers hardness of 200 or less. Note that the Vickers hardness is measured and evaluated by a nanoindentation test according to ISO 14577.

### <Step S13> Fixation of Optical Fiber

The distal end portion of the optical fiber 40 is inserted in the insertion hole H30 of the ferrule 30 and fixed with an adhesive (not shown). An inner diameter of the insertion hole H30 may define a columnar shape or any other prismatic shape such as a quadratic prism or hexagonal prism as long as a wall surface of the through hole H30 can hold the optical fiber 40. For example, a material of the ferrule 30 is a metal member of ceramic, silicon, glass, or SUS.

To prevent a stress resulting from deformation of the sleeve 50 from affecting the optical fiber 40, it is preferable that the ferrule 30 be made of a material harder than the sleeve 50, such as a material with a Vickers hardness of 400 or more.

Note that Step S13 may precede Step S11.

### <Step S14> Insertion of Ferrule

The ferrule 30, in which the optical fiber 40 is inserted, and the uncured thermosetting resin 55 are inserted in the through hole H50 of the sleeve 50. For example, the optical fiber 40 with the resin 55 being applied to a side surface of the optical fiber 40 is inserted in the through hole H50.

Note that since the gap between the sleeve 50 and the ferrule 30 is approximately in a range from 0.5 μm to 3 μm, an excess of the resin 55 is pushed out onto the upper and lower surfaces of the ferrule 30.

As shown in FIG. 2, in the optical module 1, a bottom surface of the ferrule 30 is brought into contact with the second principle surface 20SB of the wiring board 20, defining a distance d, for example, in a range from 30 μm to 100 μm between a distal end surface of the optical fiber 40 and the light emitting surface 10SA of the light emitting element 10 (passive alignment). In other words, the distance d is determined in Step S13 (a process of fixing the optical fiber 40 to the ferrule 30).

Alternatively, the distance d between the distal end surface of the optical fiber 40 and the light emitting surface 10SA of the light emitting element 10 may be defined when, as a result of vertically moving the ferrule 30 while measuring a light quantity of the optical signal guided through the optical fiber 40, the ferrule 30 reaches a position where a maximum light quantity is provided (active alignment).

### <Step S15> Plastic Deformation of Sleeve

With the distance d between the distal end surface of the optical fiber 40 and the light emitting surface 10SA of the light emitting element 10 being defined, a side surface of the sleeve 50 is held with tweezers (not shown), applying the stress F. This causes the deformation ranges D50 of the sleeve 50 to be plastically deformed with the inner surface of the sleeve 50 being crimped to the side surface of the ferrule 30, thus fixing the ferrule 30 to the sleeve 50 (so-called swaging process). In other words, such a crimping process makes outer surfaces and inner surfaces of the deformation ranges D50 of the sleeve 50 concave and convex, respectively.

In a case of using the tweezers for holding, the two deformation ranges D50A, D50B are formed at 180-degree rotational symmetric positions. To fix the ferrule 30 at a center of the sleeve 50, it is preferable that the deformation ranges D50 be defined at respective rotational symmetric positions around the center axis (optical axis O) of the sleeve 50 and four deformation ranges D50 may be defined at 90-degree rotational symmetric positions.

### <Step S16> Curing of Adhesive

For example, the resin 55, i.e., adhesive, is cured by a heat treatment at 120° C. for 30 minutes. The curing process of the resin 55 is performed with the ferrule 30 being temporarily fixed by the deformation ranges D50 of the sleeve 50.

The production method of the optical module according to the present embodiment eliminates the necessity of a special tool or the like for temporary fixation during the heat treatment, thus exhibiting a high productivity. Furthermore, since the stress resulting from deformation of the sleeve 50 is not applied to the optical fiber 40, transmission properties of the optical module are stable.

Note that the optical element in the optical module 1 is the light emitting element 10 including the light emitting portion 11. However, it goes without saying that an O/E optical module, which is a light receiving element including a light receiving portion such as a photodiode, exhibits the same effects as those of the optical module 1. In other words, the optical element only needs to emit or receive an optical signal.

### Modifications of First Embodiment

Since optical modules 1A to 1F according to modifications of the first embodiment are similar to the optical module 1 and exhibit the same effects, the same reference signs are used for functionally the same components and the explanations are omitted.

### Modification 1

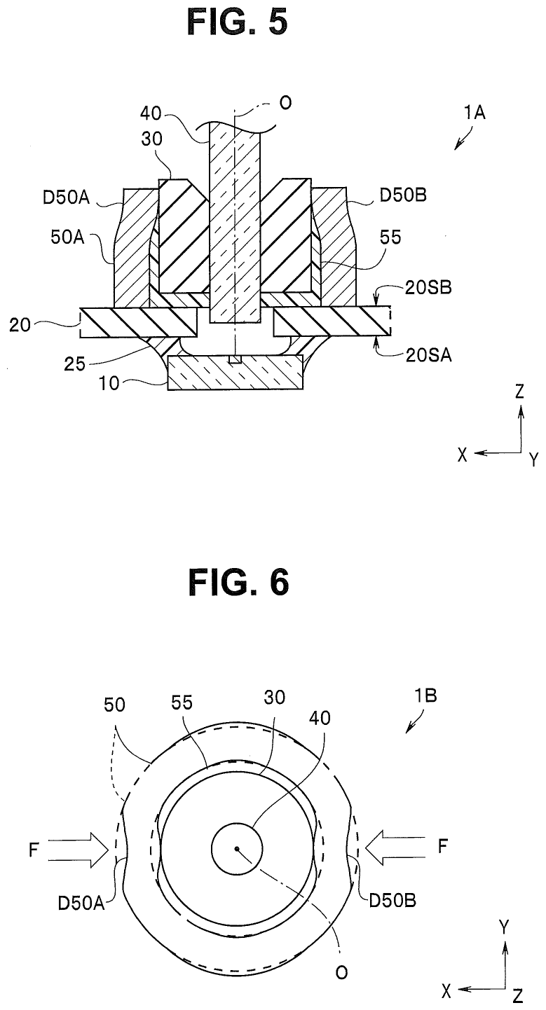

As shown in FIG. 5, the optical module 1A according to a modification 1 includes a sleeve 50A, which has a side surface with an upper portion that defines the deformation ranges D50A, D50B. In other words, the deformation ranges D50 may be defined anywhere in the side surface as long as the ferrule 30 can be fixed.

### Modification 2

As shown in FIG. 6, the optical module 1B according to a modification 2 includes a sleeve 50B, which includes portions perpendicular to the held deformation ranges D50A, D50B. The portions have inner surfaces and outer surfaces that are moved closer to the optical fiber 40 as a whole with the stress F resulting from holding both opposite side surfaces, forming deformation ranges having concave outer surfaces and convex inner surfaces with respect to surfaces of other portions. The sleeve 50B is thus plastically deformed substantially in an oval shape when seen along a Z-axis. In other words, as long as the ferrule 30 is fixed by the deformation ranges D50, any other portion of the sleeve may be plastically deformed.

### Modification 3

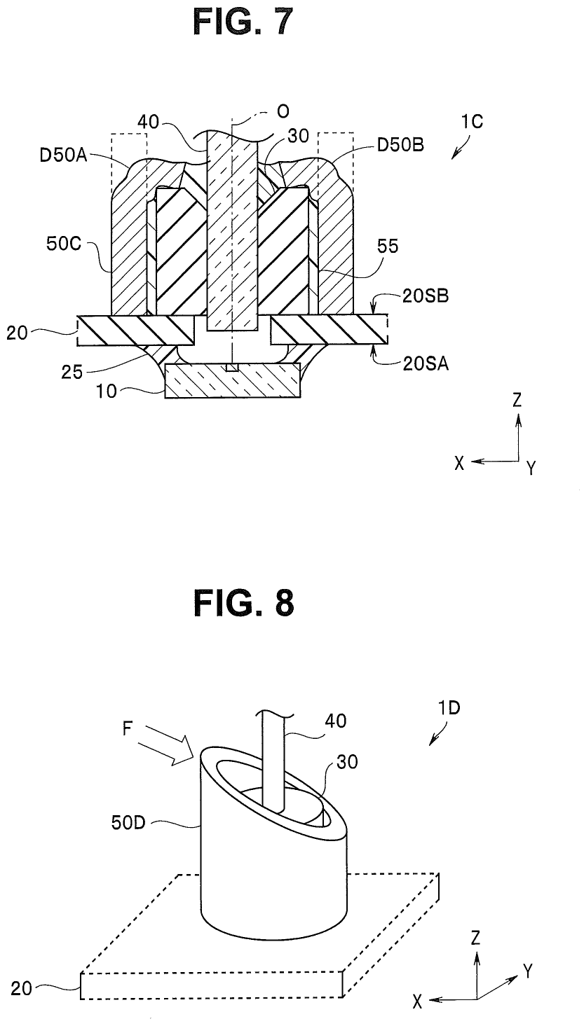

As shown in FIG. 7, the optical module 1C according to a modification 3 includes a sleeve 50C with a length (Z-axis dimension) longer than a length of the ferrule 30.

The deformation ranges D50A, D50B of the sleeve 50C have inner surfaces that are in contact with a corner portion of the upper surface, that is, a corner portion of a base end portion, where the upper surface and the side surface of the ferrule 30 border each other. As the sleeve 50C is deformed, the bottom surface of the ferrule 30 is pressed against and, consequently, reliably in contact with the second principle surface 20SB of the wiring board 20.

Furthermore, an excess of the resin 55 is prevented from spreading around as being retained between the upper surface of the ferrule 30 and an upper portion of the sleeve 50C.

Note that in a case of the passive alignment of the ferrule 30, the inner surfaces of the deformation ranges D50 may be in contact with the upper surface of the ferrule 30.

In other words, the inner surfaces of the deformation ranges D50 of the sleeve only need to be in contact with the upper surface of the ferrule or with the corner portion of the ferrule, where the upper surface and the side surface border each other, for fixation of the ferrule.

### Modification 4

As shown in FIG. 8, the optical module 1D according to a modification 4 includes a sleeve 50D with an upper surface that is a slanted surface, the sleeve 50D having a portion with a length (Z-axis dimension) longer than the length of the ferrule 30.

As being pressed with the stress F, the portion of the sleeve 50D longer than the ferrule 30 is brought into contact with the upper surface and the corner portion of the ferrule 30.

The sleeve 50D is deformable with a smaller stress F, facilitating the production.

### Modification 5

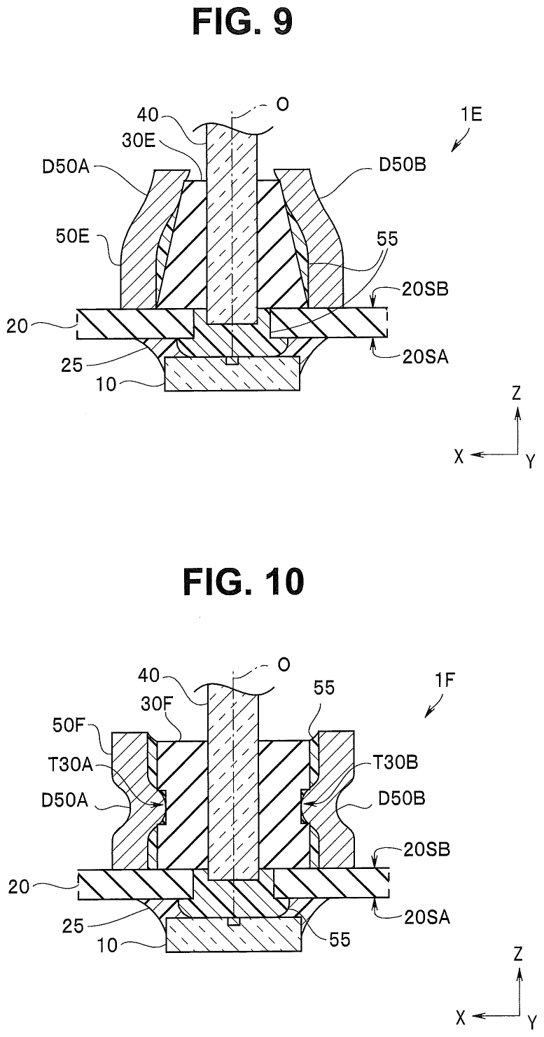

As shown in FIG. 9, the optical module 1E according to the modification 3 includes a substantially conical ferrule 30E with a flattened upper surface, which has a trapezoidal cross section. An upper portion of a sleeve 50E is plastically deformed, fixing the ferrule 30E.

In other words, the ferrule may be in any shape, such as substantially in a rectangular parallelepiped or a cone, as long as the ferrule can be fixed by plastic deformation of the sleeve. Note that the sleeve 50E in a conical shape is allowed to be positioned with a higher accuracy if an inner diameter of a lower opening of the sleeve 50E is defined to be the same as a contour of the ferrule.

For the optical module 1E, the resin 55 is transparent and also provided to fill a light path between the light emitting element 10 and the optical fiber 40. Since the transparent resin 55 is provided to fill the light path as a refractive index matching material, the optical module 1E exhibits a high transmission efficiency due to prevention of interface loss and interface reflection. Note that it goes without saying that filling the light path with the transparent resin 55 is also preferable for the optical module 1, etc.

### Modification 6

As shown in FIG. 10, the optical module 1F according to the modification 3 includes a ferrule 30F, a side surface of which is provided with recessed portions T30A, T30B where the inner surfaces of the deformation ranges D50A, D50B of a sleeve 50F are in contact.

Since the deformation ranges D50 are fitted in the recessed portions T30 of the side surface, the ferrule 30F is more firmly fixed to the sleeve 50F.

The recessed portions T30 may be formed by machining or etching. Alternatively, the recessed portions T30 may each be a groove surrounding the side surface of the ferrule or a slit penetrating between the upper surface and the lower surface of the ferrule.

### Second Embodiment

Since an optical module 1G according to a second embodiment is similar to the optical module 1 and exhibits the same effects, the same reference signs are used for functionally the same components and the explanations are omitted.

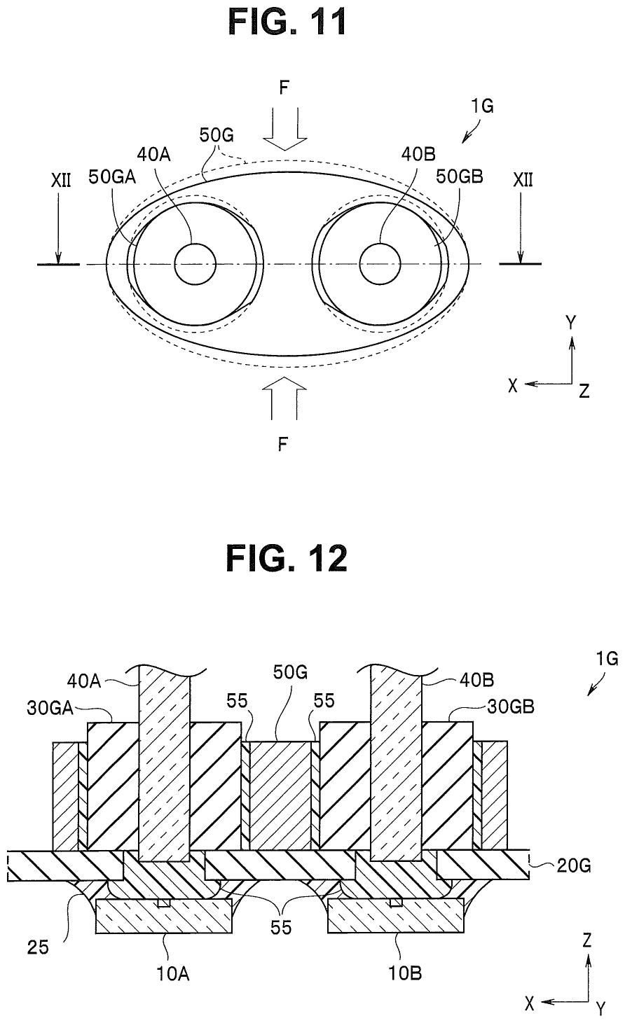

As shown in FIGS. 11 and 12, the optical module 1G includes a plurality of optical fibers 40A, 40B, a plurality of light emitting elements 10A, 10B, and a plurality of ferrules 30GA, 30GB. The plurality of ferrules 30GA, 30GB are inserted in a single sleeve 50G.

In other words, the sleeve 50G, which is a metal cylinder, is provided with a plurality of through holes H50A, H50B. The sleeve 50G, which is in an oval shape in plan view, is held at side surfaces defined in a direction (Y-direction) perpendicular to an alignment direction (X-direction) of the plurality of through holes H50A, H50B with application of the stress F. The sleeve 50G is thus plastically deformed, simultaneously fixing the plurality of ferrules 30GA, 50GB.

Although including the plurality of optical fibers, the optical module 1G is compact and easy to produce.

### Modifications of Second Embodiment

Since optical modules 1H to 1I according to modifications of the second embodiment are similar to the optical modules 1, 1G and exhibit the same effects, the same reference signs are used for functionally the same components and the explanations are omitted.

### Modification 1 of Second Embodiment

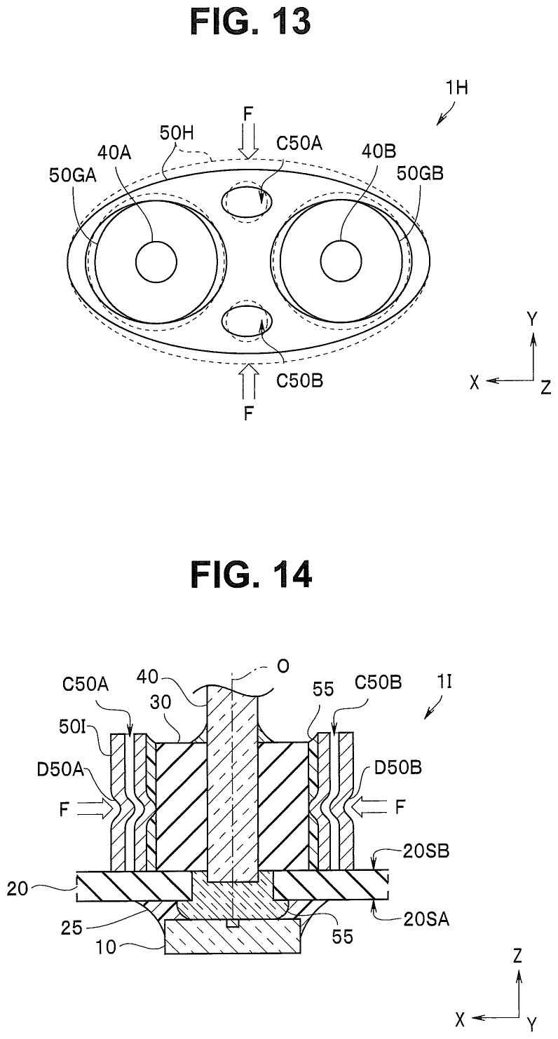

As shown in FIG. 13, a sleeve 50H of an optical module 1H according to the present embodiment, which is similar to the sleeve 50G of the optical module 1G according to the second embodiment, is provided with cavities C50A, C50B between an outer surface and an inner surface of the sleeve 50H. The deformation ranges D50 are deformed with the stress F applied by holding the outer surfaces of the cavities C50A, C50B.

The cavities C50A, C50B allow the deformation ranges of the sleeve 50H to be plastically deformed with a smaller stress F than the stress for the sleeve 50, further facilitating the production.

### Modification 2 of Second Embodiment

As shown in FIG. 14, a sleeve 501 of the optical module 1I according to the present embodiment, which is similar to the sleeve 50 of the optical module 1 according to the first embodiment, is provided with the cavities C50A, C50B between an outer surface and an inner surface of the sleeve 501. The deformation ranges D50 are deformed with the stress F applied to the outer surfaces of the cavities C50A, C50B.

The sleeve 501 is plastically deformed with a smaller stress F than the stress for the sleeve 50, further facilitating the production.

### Third Embodiment

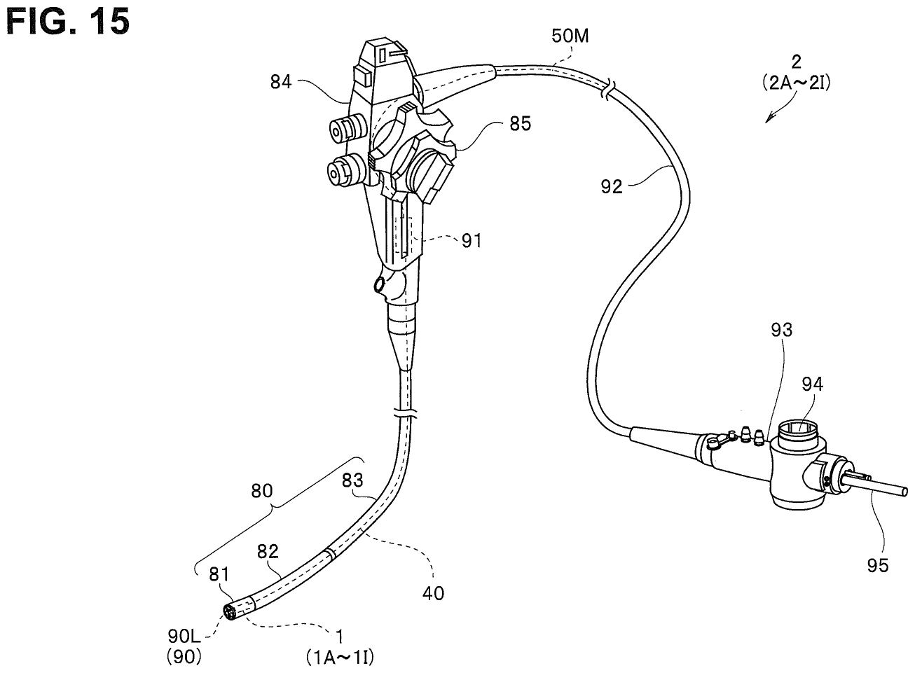

As shown in FIG. 15, an endoscope 2 (2A to 2I) according to the present embodiment includes an insertion portion 80, an operation portion 84 disposed near a proximal end portion of the insertion portion 80, a universal cord 92 extending from the operation portion 84, and a connector 93 disposed near a proximal end portion of the universal cord 92.

The insertion portion 80 includes a rigid distal end portion 81, a bending portion 82 for changing an orientation of the distal end portion 81, and an elongated bendable flexible portion 83, which are connected in series.

The distal end portion 81 is provided with an image pickup optical unit 90L, an image pickup device 90, and the E/O optical module 1 that converts an image pickup signal (electric signal) from the image pickup device 90 into an optical signal. The image pickup device 90 may be a CMOS (complementary metal oxide semiconductor) image sensor or a CCD (charge coupled device).

The operation portion 84 is provided with an angle knob 85 for operating the bending portion 82 and an O/E optical module 91 that converts an optical signal into an electric signal. The connector 93 includes an electric connector portion 94 connected to a processor (not shown) and a light guide connection portion 95 connected to a light source. The light guide connection portion 95 is connected to an optical fiber bundle that guides an illumination light to the rigid distal end portion 81. Note that the connector 93 may be integral with the electric connector portion 94 and the light guide connection portion 95.

In the endoscope 2 (2A to 2I), the image pickup signal is converted into an optical signal by the E/O optical module 1 (1A to 1H) provided at the distal end portion 81 and transmitted to the operation portion 84 through the thin optical fiber 40 inserted in the insertion portion 80. The optical signal is again converted into an electric signal by the O/E optical module 91 provided at the operation portion 84 and transmitted to the electric connector portion 94 through a metal wiring 50M inserted in the universal cord 92. In other words, the signal is transmitted within the insertion portion 80 with a small diameter through the optical fiber 40, whereas the signal is transmitted within the universal cord 92, which is not to be inserted in a body and thus has less limitations of the outer diameter, through the metal wiring 50M thicker than the optical fiber 40.

Note that in a case where the optical module 91 is disposed in a vicinity of the electric connector portion 94, the optical fiber 40 may be inserted in the universal cord 92 to the vicinity of the electric connector portion 94. In a case where the optical module 91 is disposed in the processor, the optical fiber 40 may be inserted to the connector 93.

Since the endoscope 2 (2A to 2I) enables optical signal transmission such as transmission of an optical signal through the thin optical fiber 40 instead of electric signal transmission, the insertion portion 80 is thinned and less invasive.

The present invention is by no means limited to the above embodiments, modifications, etc., but a variety of modifications, combinations, and applications are possible without departing from the scope of the invention.

What is claimed is:

1. An optical module comprising:
an optical fiber configured to transmit an optical signal;
a ferrule with an insertion hole in which the optical fiber is inserted;
a sleeve with a through hole in which the ferrule is inserted;
an optical element configured to emit or receive the optical signal; and
a wiring board having a first principle surface and a second principle surface, the optical element being disposed on the first principle surface, the sleeve being disposed on the second principle surface, wherein
the sleeve has a deformation range where an outer surface is concave and an inner surface defining the through hole is convex, the inner surface of the deformation range being in contact with the ferrule, and
the sleeve includes a metal cylinder with a resin provided to fill a space between the metal cylinder and the ferrule.

2. The optical module according to claim 1, wherein
the sleeve includes the deformation range at two opposite positions with respect to a center axis.

3. The optical module according to claim 1, wherein
the inner surface of the deformation range is in contact with a side surface of the ferrule.

4. The optical module according to claim 3, wherein
the side surface of the ferrule includes a recessed portion where the inner surface of the deformation range is in contact.

5. The optical module according to claim 1, wherein
the inner surface of the deformation range is in contact with a corner portion of the ferrule where an upper surface and a side surface of the ferrule border each other.

6. The optical module according to claim 1, further comprising
a plurality of optical fibers including the optical fiber, a plurality of optical elements including the optical element, and a plurality of ferrules including the ferrule, wherein
the sleeve is provided with a plurality of through holes including the through hole, the plurality of ferrules each being inserted in corresponding one of the plurality of ferrules.

7. The optical module according to claim 1, wherein
the deformation range of the sleeve is provided with a cavity between the outer surface and the inner surface.

8. The optical module according to claim 1, wherein
the wiring board is provided with a hole that defines a light path, and the resin is transparent and is provided to fill in the hole and between the optical element and the optical fiber.

9. An endoscope comprising:
an insertion portion; and
an optical module provided at the insertion portion, the optical module including:
an optical fiber configured to transmit an optical signal;
a ferrule with an insertion hole in which the optical fiber is inserted;
a sleeve with a through hole in which the ferrule is inserted;
an optical element configured to emit or receive the optical signal; and
a wiring board having a first principle surface and a second principle surface, the optical element being disposed on the first principle surface, the sleeve being disposed on the second principle surface, wherein
the sleeve has a deformation range where an outer surface is concave and an inner surface defining the through hole is convex, the inner surface of the deformation range being in contact with the ferrule, and
the sleeve includes a metal cylinder with a resin provided to fill a space between the metal cylinder and the ferrule.

10. A production method of an optical module, the optical module including: an optical fiber configured to transmit an optical signal; a ferrule with an insertion hole;
a sleeve including a metal cylinder with a through hole; an optical element configured to emit or receive the optical signal; and a wiring board having a first principle surface and a second principle surface, the optical element being disposed on the first principle surface, the sleeve being disposed on the second principle surface, the method comprising:
disposing the optical element and the sleeve on the wiring board;
fixing the optical fiber to the ferrule after the optical fiber is inserted in the through hole of the ferrule;
inserting the ferrule in which the optical fiber is inserted and an uncured adhesive in the through hole of the sleeve;
forming a deformation range where an outer surface is concave and an inner surface defining the through hole is convex by plastic deformation of the sleeve;
fixing the ferrule to the sleeve by bringing the ferrule into contact with the inner surface of the deformation range; and
curing the adhesive.
